(19) United States
(12) Patent Application Publication
(10) Pub. No.: US 2011/0165677 A1
(43) Pub. Date: Jul. 7, 2011

(54) METHOD FOR REDUCING DEPOSITS DURING THE CULTIVATION OF ORGANISMS

(75) Inventors: Helmut Brod, Koln (DE); Björn Frahm, Erkrath (DE); Joerg Kauling, Koln (DE)

(73) Assignee: Bayer Technology Services GMBH, Leverkusen (DE)

(21) Appl. No.: 13/062,556

(22) PCT Filed: Sep. 17, 2009
(86) PCT No.: PCT/EP2009/006722

§ 371 Date: Mar. 7, 2011

(30) Foreign Application Priority Data
Sep. 26, 2008 (DE) 102008049120.9

(51) Int. Cl. C12N 5/02; C12M 3/02

(52) U.S. Cl. 435/394; 435/420; 435/297.1

(57) ABSTRACT

The invention relates to a process for reducing the amount of deposits during the culture of organisms, and in particular of cell cultures, where these have a tendency towards agglomeration or adhesion to the bioreactor and to its elements, or where these are readily susceptible to agglomeration or adhesion of cell debris or of substances.

This is a 371 of PCT/EP2009/006722 filed Sep. 17, 2009, which claims priority of German application no. 10 2008 049 120.9 filed Sep. 26, 2008.

The invention relates to a process for reducing the amount of deposits during the culture of cells or organisms, and in particular of cell cultures, where these have a tendency towards agglomeration or adhesion to the bioreactor and to its elements, or where these are readily susceptible to agglomeration or adhesion of cells, cell debris or of substances.

Culture of human, animal and plant-derived cells has achieved great importance in the production of biologically active substances and of pharmaceutically active products. Culture of cells is frequently carried out in free suspension in a nutrient medium, and this is a particularly demanding process because, unlike microorganisms, the cells are very easily damaged by mechanical shear and by inadequate supply of oxygen and of nutrients (see, for example, H.-J. Henzler 2000. Particle Stress in Bioreactors. Adv. Biochem. Eng./Biotechnol. 67:35-82 or J. G. Aunis, H.-J. Henzler 1993. Aeration in Cell Culture Bioreactors, in: Biotechnology, Second, Completely Revised Edition, Volume 3: Bioprocessing: 219-281, VCH Wiley or Untersuchungen zum extrazellulären and intrazellulären Sauerstofftransfer [Investigations into extracellular and intracellular oxygen transfer], doctoral thesis by Oliver Schweder, Faculty of Natural Sciences in the University of Hanover, 2006). The solubility of oxygen in the nutrient medium is so low that without continuous oxygen supply the cells would rapidly suffer from oxygen shortage; this is quite different from the situation with nutrients whose concentration in the nutrient medium is such that they do not need to be continually supplemented. Another factor of similar importance, alongside adequate oxygen supply, is carbon dioxide removal.

Human, animal and plant-derived cell lines are mostly cultured in batches. A disadvantage of this is that substrate concentrations, product concentrations and biomass concentrations are constantly changing, making it difficult to achieve an optimum supply to the cells. Furthermore, at the end of the fermentation process there is an increased concentration of by-products, e.g. the lysis products of dead cells, and large amounts of resource are mostly needed to eliminate these during subsequent treatment processes. It is therefore preferable to use continuously operated bioreactors, and this is particularly the case when unstable products are produced, examples being those that can be damaged via proteolytic attack. When compared with batch culture systems, continuous bioreactors can achieve higher cell densities and, associated therewith, higher productivity.

Some cell lines have the property of preferentially forming agglomerates and/or adhering to the internal regions of a culture vessel/bioreactor, or of causing/promoting deposition of cell debris or substances (e.g. proteins) on the internal regions of the culture vessel (see, for example, EP0242984B1). This is generally disadvantageous, since the ability of elements within the bioreactor to function is sometimes considerably restricted or even eliminated, examples being membranes for purposes of gas transfer, or probes.

In conventional systems, the deposition of debris is a primary reason for the requirement to reduce cell density and for the premature termination of culture of the cell lines involved. The deposition of cells, debris and/or proteins on probes or on other measurement/analysis equipment in conventional systems moreover causes malfunction or failure of the said equipment, and it is usually impossible to correct or compensate for this during continuing operation of the bioreactor, and the result can therefore be premature termination of the culture process.

The literature reveals a long history of problems in (cell culture) fermentation processes relating to deposits in the bioreactor and to the adhesion of cells and cell debris, in particular on the membrane tubes, and also in relation to formation of cell agglomerates.

In order to reduce the amount of deposits, EP0242984B1 provides a double-walled fermenter with a spiral stirrer, the stirrer blades of which extend almost as far as an interior (semipermeable) wall of the fermenter. The movement of the stirrer blades in the vicinity of the interior wall causes turbulence phenomena, which are intended to inhibit the deposition of cells/cell residues/cell products on the interior wall and thus inhibit fouling. A disadvantage of the fermenter described is that cell cultures can be damaged by the turbulence phenomena. It is moreover known (see, for example, EP0422149B1) that agitator units, in particular the stirrer blades described in EP0242984B1, produce high shear forces which can damage cell membranes, in particular of cells having no cell walls.

EP1935973A1 describes a culture apparatus for aquatic organisms which does not require an agitator unit. Gas (oxygen for supply to the organisms) is introduced in the vicinity of the outlet at the bottom of the vessel and generates a flow which affects all of the culture medium within the vessel, and is intended to achieve complete mixing of the constituents of the culture medium and of the, if appropriate, free-floating culture organisms. The arrangement in one particular embodiment has a single nozzle centrally within the outlet, and a partition introduced within the vessel in such a way that the inflow of gases gives a flow in a clearly defined direction, composed of an upwards and downwards movement of the nutrient liquid around the partition. It is known (see, for example, EP0422149B1) that high shear forces are active during production and collapse of gas bubbles, and that these forces can cause cell damage. Gas bubbles moreover lead to formation of foam. Formation of foam should be avoided, however, since cells have a tendency to float with the foam, and the culture conditions that they encounter within the foam layer are inadequate. The use of antifoaming agents can lead to cell damage or reduced yields during treatment processes or to a need for increased resource in the treatment processes. Another factor is that use of gas-introduction methods involving large bubbles cannot ensure adequate oxygen supply to shear-sensitive cells except at relatively low cell densities (H.-J. Henzler: “Verfahrentechnische Auslegungsunterlagen für Rührbehälter als Fermenter” [Process-technology design documentation for stirred fermenters] Chem. Ing. Tech. 54 (1982) No. 5 pp. 461-476, H.-J. Henzler, J. Kauling: “Oxygenation of cell cultures” Bioprocess Engineering 9 (1993) pp. 61-75, “Mischen and Rühren” [Mixing and agitation], edited by M. Kraume, WILEY-VCH 2003). Scale-up, to an industrial scale bioreactor, of a gas-introduction process involving large bubbles is difficult. The culture apparatus described in EP1935973A1 is therefore unsuitable for cultivation of a wide variety of organisms on an industrial scale.

Bubble-free gas introduction solves the problems by using gas transfer by way of an immersed membrane area. The gas-introduction process here uses continuous-surface or open-pore membranes. By way of example, the arrangement has these within the liquid which is moved by an agitator. It is possible, for example, to wind membranes in the form of tubes on cylindrical basket stators (H.-J. Henzler, J. Kauling: “Oxygenation of cell cultures” Bioprocess Engineering 9 (1993) pp. 61-75, EP0172478B1, EP0240560B1). In order to accommodate large oxygen-exchange areas, the tubes are placed close to one another with minimum separation. In the context of porous polymers, silicone has achieved wide acceptance as tube material. Reasons for this are high gas permeability, high thermal stability and tube properties homogeneously distributed over the length of the tube segments of up to approx. 70 m, these properties also being retained after sterilization. However, between the tubes, and between the stator and the tubes, there are problematic dead volumes where deposits can easily form. Continuing deposition of substances on the silicone tubes themselves leads to increasing deterioration of gas transfer, e.g. for purposes of supply of oxygen to cells or in the removal of carbon dioxide. The silicone tube is generally discarded after a single use.

A disadvantage of the membrane gas-introduction process described is also the comparatively small mass-transfer coefficient (H.-J. Henzler, J. Kauling: “Oxygenation of cell cultures” Bioprocess Engineering 9 (1993) pp. 61-75). In order to achieve high mass-transfer rates, it is necessary to install an appropriately large membrane area within the bioreactor. However, this requires large amounts of resource for design and operation (assembly, sterilization, cleaning, generation of regions with inadequate mixing, etc.) and leads to increased dead volumes. One option is to increase power consumption. The mass-transport coefficient is dependent on power consumption, and this can therefore be used to raise the mass-transfer rate. However, the potential gain is limited by the resultant shear load on the cells resulting from the higher power consumption.

WO2007098850(A1) describes a process and an apparatus for gas introduction into liquids, in particular liquids used in biotechnology, and specifically cell cultures; in this process and apparatus, gas transfer takes place by way of one or more immersed membrane areas of any desired type (e.g. tubes), where the membrane area executes any desired rotary oscillating movement within the liquid. The movement can be optimized in such a way as to give ideal flow onto the membrane area. The mass-transport coefficient is dependent on the flow onto the membrane area, and it is thus possible to achieve improved oxygen supply. A further advantage of the rotary oscillating movement of the membrane area is that there is no requirement for a separate agitation or mixing unit to produce flow onto the membrane area.

While the rotary oscillating movement described in WO2007098850(A1) for the membrane area achieves an improvement in oxygen supply and a reduction in the shear forces, in comparison with the static membranes which are described in EP0172478B1 and EP0240560B1 and onto which flow is directed by an agitator unit, there is a risk in WO2007098850(A1) that the movement of the membrane tubes through the culture medium traps particles which either become fixed to the membrane tubes or pass along the membrane tubes into the dead volumes between the membrane tubes, where they cause deposits.

WO86/07604A1 describes an air-lift fermenter. The proposal is to use flocculants during the culture of animal cells in order to separate cell debris from the product stream. The heavy particles removed by flocculation sink into a turbulence-free zone of the air-lift fermenter, and here they can be withdrawn. The use of flocculants can reduce the amount of deposits but cannot provide long-lasting deposit prevention. A risk inherent in the use of flocculants together with rotary-oscillating membrane areas for supply of oxygen is that particles removed by flocculation are trapped and transported into dead volumes where they can become permanently fixed. The use of flocculants is moreover not appropriate in the culture of all cell lines, since flocculants can have an adverse effect on the physiology of the cells, and excess flocculant may have to be removed from the product.

Starting from the prior art described, an object is to provide a process for reducing the amount of deposits during the culture of cells or organisms, and in particular during the culture of cell cultures, where these have a tendency towards agglomeration or adhesion to the bioreactor and to its elements, or where these are readily susceptible to agglomeration or adhesion of cells, cell debris or of substances. The desired process should ensure ideal supply of nutrients to the organisms, in particular in respect of gas transfer, e.g. involving oxygen. The process should not require the use of additional shear forces leading to destruction of cells and thus to reduction of productivity. The desired process should not require the use of chemicals (e.g. flocculant), the aim being to avoid additional stress on the organisms and to avoid any relatively high use of resources for product isolation. The desired process should in particular reduce the amount of deposits leading to reduced gas transfer, e.g. reduced oxygen supply. The desired process should be easy to execute and inexpensive.

Surprisingly, it has been found that the agglomeration and/or deposition of cells, debris and/or proteins, in particular on the elements for gas transfer, e.g. for oxygen supply, but also on all other areas, and also probes, can be significantly reduced or indeed prevented by a membrane area which is immersed in the culture medium for purposes of gas supply and which executes a discontinuous movement within the culture medium.

The present invention therefore provides a process for reducing the amount of deposits during the culture of cells and organisms, and in particular of cell cultures, where these have a tendency towards agglomeration or adhesion to the bioreactor and to its elements, or where these are readily susceptible to agglomeration or adhesion of cells, cell debris or of substances, characterized in that a membrane area immersed in the culture medium for purposes of gas transfer executes a discontinuous movement.

The discontinuous movement significantly reduces or prevents the agglomeration and/or deposition of cells, debris and/or proteins, particularly on the membranes, but also on all other contact areas, and also probes, giving a higher level of gas transfer across the membranes, and this also applies over a prolonged period.

This gives an increase in cell density and therefore in product yield, and prolongs the maximum running time of the process.

The term “movement” generally means a process in which a moving body (here the membrane area) experiences a change of its arrangement within space. It is possible here that the entire body moves (translation) or that only portions of the body move, e.g. via bending of the body (vibration). It is also possible that the movement of the body consists is rotation. Combinations of translation, vibration and rotation are also possible.

The term “discontinuous movement” means a movement which does not proceed uniformly over a given period of time. An example of a discontinuous movement is the movement of a pendulum. Over the time represented by one period, e.g. beginning with maximum deflection of the pendulum towards the right-hand side, the pendulum first executes an accelerating movement towards the left-hand side, until it has maximum velocity when it is vertical. The pendulum then undergoes gradual retardation until, with maximum deflection towards the left-hand side, it is stationary for a moment before again accelerating, reaching maximum velocity when vertical and again undergoing retardation until it has again reached its starting position (maximum deflection towards the right-hand side). In contrast to this, a continuous movement is a movement which is uniform over a given period of time. An example of a continuous movement is the rotation of a stirrer element with constant angular velocity around a fixed axis of rotation.

It is preferable that the membrane area executes a discontinuous movement with a reversal of movement, i.e. that the membrane area first executes a first movement of any desired type in a first direction, before becoming stationary and then executing a second movement of any desired type in another direction, preferably in the direction opposite to the first direction. The first movement and the second movement can be completely different from one another. However, it is preferable that the second movement is related to the first movement via mirror symmetry, point symmetry and/or rotational symmetry.

In one preferred embodiment of the process according to the invention, the membrane area executes an oscillating movement. The term “oscillating” means a process which is repeated regularly and uniformly, and this means that the process according to the invention is preferably characterized in that there is a period of time, hereinafter called a period, within which the membrane area completes any desired first movement, and that subsequent movements are copies of the first movement featuring the same chronological sequence of accelerations and velocities as the first movement. The movement described above for a pendulum is an example of an oscillating movement.

It is particularly preferable that the membrane area executes a rotary oscillating movement. In a rotary oscillating movement, the membrane area first moves (rotates) in one direction of rotation, where the type of movement can be as desired. One example is the acceleration of the membrane area with a certain angular acceleration until a particular angular velocity has been reached, with which the membrane area then moves for a certain time. The membrane area is then retarded with a defined retardation until it becomes stationary. There then follows, if appropriate after a defined stationary time, the movement in the other direction of rotation. This movement can be a mirror reflection of that described above or can be of some other type. Another movement that is to be understood as a rotary oscillating movement is a movement in which the membrane area is first accelerated in one direction and rotates in this prescribed direction with constant velocity for a time t which is greater than or equal to zero, and is then retarded (whereupon the membrane area can become stationary or can also rotate further in the same direction with a small angular velocity) and then is again accelerated in the same direction.

The movement is preferably executed in such a way that the membrane area first rotates in one direction and, after a prescribed time, rotates in the opposite direction.

In one preferred embodiment of the process according to the invention, the movement of the membrane area is a rotary oscillating movement with reversal of direction of rotation and with minimum stationary times at the points of reversal of direction of rotation. Minimum stationary time means that the reversal of direction of rotation takes place without any technical/avoidable delay, i.e. that immediately after reaching a point of reversal of direction of rotation the membrane area experiences an acceleration in the direction opposite to the prior direction. The preferred embodiment is further characterized in that, starting from a point of reversal of direction of rotation, the membrane area is accelerated for a definable period of time with constant angular acceleration and then, on reaching a maximum velocity, the membrane area is in turn retarded with a constant angular retardation, until the membrane area reaches the second point of reversal of direction of rotation (movement phase 1). There then follows a movement phase 2 which is a mirror image of movement phase 1. It is preferable that the constant angular acceleration and angular retardation are numerically equal. The preferred embodiment of the process according to the invention is characterized in that no movement phase with a constant angular velocity occurs.

In one preferred embodiment of the process according to the invention, flow impacts the membrane area tangentially as a consequence of the discontinuous movement within the culture medium. The tangential impact of flow ensures effective gas transfer between membrane area and culture medium (oxygen supply, carbon dioxide removal).

The term “membrane area” means an area through which a gas, in particular oxygen, can be introduced in dissolved form or in the form of fine bubbles into a liquid, and/or a gas can be removed from the liquid. The term “fine gas bubbles” means gas bubbles which have little tendency towards coalescence within the culture medium used.

Examples of suitable membrane areas are specific sintered bodies made of metallic and ceramic materials, and filter sheets or laser-perforated sheets, where these have pores or holes with a diameter which is generally smaller than 15 μm. The membrane areas preferably take the form of hollow bodies, e.g. pipes, through which the gas can flow. Small open-pipe gas velocities of less than 0.5 m h⁻¹ produce very fine gas bubbles which have little tendency towards coalescence in the fluids normally used in cell culture.

Other suitable membrane areas are membrane tubes. Membrane tubes are flexible pipe-shaped structures which are permeable to gases, such as oxygen and carbon dioxide. Examples that may be mentioned are hollow-filament membranes made of microporous polypropylene, such as those described by way of example by H. Bünterneyer et al. in Chem.-Ing.-Tech. 62 (1990), No. 5, pp. 393-395. It is equally possible to use silicone tubes, as described by way of example in the following documents: H.-J. Henzler, J. Kauling: “Oxygenation of cell cultures” Bioprocess Engineering 9 (1993) pp. 61-75, EP 1948780, WO07/051,551A1, WO07/098,850A1.

The membrane areas used preferably comprise non-porous silicone tubes. These preferably lie within the range of internal diameter ˜1 mm with external diameter ˜1.4 mm to internal diameter ˜2 mm with external diameter ˜3 mm. The tube diameter and total tube length parameters should be selected in such a way as to ensure adequate mass transfer for the application. Mass transfer of materials is determined inter alia by the ratio of membrane surface area to reactor liquid volume (volume-specific mass-transfer area). Familiar values here are from 25 m⁻¹ to 45 m⁻¹ for animal cell cultures. In the process according to the invention, the volume-specific mass-transfer area achieves values of from 0.1 m⁻¹ to 150 m⁻¹, preferably from 1 m⁻¹ to 100 m⁻¹ and particularly preferably from 5 m⁻¹ to 75 m⁻¹.

In one preferred embodiment of the process according to the invention, the membrane area has been attached to a rotatably mounted rotor which can be moved within the interior of a container, e.g. of a bioreactor. The design of the rotor is such that, within the interior of the bioreactor, it can support at least one membrane area, e.g. tubes, cylinders, modules, etc. The rotor is preferably used to execute a rotary oscillating movement. For this, the rotatably mounted rotor can by way of example be provided with a rotary oscillating movement via a drive, from outside the bioreactor. The required drive torque can be transferred from the drive to the rotor within the interior of the reactor by way of a magnetic coupling, or the rotor shaft can be passed by way of a rotating seal through the casing of the bioreactor and coupled directly to the drive. The use of a magnetic coupling is particularly advantageous for reasons of sterilization technology, because it separates sterile and non-sterile spaces in a clear manner from one another, without any rotating seal.

The power provided from the motor in the form of drive for generating a rotary oscillating movement must be sufficient to carry out an oscillating movement of the rotor with the prescribed sequence of movement, despite the inertia due to the mass of the rotor and of the culture medium. The inertia due to the mass of the rotor is therefore a decisive factor for the design of the drive, as also is the force that the culture medium exerts on the rotor. Given an adequate rotation rate of the motor, a gearbox makes it possible to provide the required torque. An example of a drive configuration that can be used is an eccentric drive. An eccentric drive converts the uniform rotation of a conventional drive motor to a rotary oscillating movement at the drive shaft. Freely programmable position drives are another possible drive configuration for the apparatus according to the invention, an example being a stepping motor. The advantage of these freely programmable drive systems is that the rotary oscillating movement of the membrane area can be adapted to be appropriate to the requirements of the process within wide limits, whereas an eccentric drive generally has only restricted possibilities for adjustment.

Drive parameters such as rotation rate, torque and gearbox reduction ratio can be freely selected for the particular application and are scale-dependent. For applications in the biotechnology sector, the parameters are usually such as to give a volume-specific power consumption of from 0.01 W per m⁻³ up to 4000 W per m⁻³ of liquid volume, preferably around 1000 W per m⁻³.

For cell cultures, the volume-specific power consumption is usually from 0.01 to 100 W per m⁻³.

For the cell culture application, the parameters should moreover be such as to give maximum relative velocities of 1 m s⁻¹ between rotor and culture medium.

In order to absorb the stresses from the transmission/rotor connection, the transmission is usually connected to the rotor by way of any desired torsionally stiff coupling which absorbs small shaft misalignments or any small amount of non-alignment of the shafts.

Advantageously, the design of the apparatus for attaching one or more membrane areas can readily be adapted to the particular conditions found in cell cultures, e.g. cell agglomeration. By way of example, this can be achieved via the nature and arrangement of the membrane areas.

The rotor preferably has from 1 to 64 rotor arms, preferably from 2 to 32 and particularly preferably from 4 to 16, on which one or more membrane areas can be attached.

In one particular design of the apparatus, a rotor arm is formed by two winding arms. The membrane area, preferably the membrane tubes, is/are wound horizontally or vertically with regular or irregular separation on the said winding arms.

If the rotor now rotates, the membrane tubes are moved through the culture medium within the reactor and thus tangentially impacted by flow. Surprisingly, it has been found that the impact of the flow does not, as the person skilled in the art would assume, cause particles in the solution to be trapped by the membrane areas and fixed or transported into dead volumes (of these), with resultant deposits. Surprisingly, it has been found that a discontinuous movement, preferably a rotary oscillating movement, reduces the amount of deposits when comparison is made with a statically arranged membrane area where, if appropriate, an additional agitator unit brings about flow of culture medium onto the material.

With respect to flow onto the membrane tubes, it should be noted that, for the same angular velocity, this flow generally improves as a function of the position of the membrane tube as radial distance from the rotor shaft increases. The reason for this is that the peripheral velocity increases to the same extent. It is preferable to install a maximum number of membrane tubes with maximum distance from the centre, with good flow onto the material. One way of complying with this requirement consists in increasing the number of rotor arms around the shaft. However, an increase in the number of the arms has an adverse effect not only on the mixing process but also on flow onto the membrane (creation of compartments where mixing is less effective, between the arms). Another factor is that the increasing number of arms makes operation of the rotor more difficult during winding and unwinding of the tubes, and also during installation and dismantling. As the number of the arms becomes greater it also becomes more difficult to secure the arms to the shaft, for reasons of space.

The supply to the discontinuously moving membrane area for the introduction and dissipation of gas preferably takes place from the stationary environment, e.g. from the top cover of the reactor, using a rotary seal with the aid of flexible tubes. Rotary seals are mostly undesirable in cell culture technology since they can cause difficulties with cleaning and sterilization. The process according to the invention, with reversal of movement, has a clear advantage here in comparison with a process without reversal of the direction of movement: without reversal of the direction of movement the tubes would be subject to constantly increasing torsion as rotation increased and finally would tear. In the case of a movement with reversal of movement, e.g. in the case of membrane areas undergoing rotary oscillation, the to-and-fro movement means that no net torsion of the flexible tubes occurs. A precondition is naturally that the to-and-fro movement is designed in such a way that on conclusion of one period of the movement the location of the membrane area is at the starting point of the movement.

Another advantage of the apparatus with wound membrane tubes is that the tension of the membrane area, e.g. of the membrane tubes, can be varied. The ideal tension results inter alia from the following parameters: the pressure of the gas or gas mixture flowing into the space within the membrane area, the pressure of the gas or gas mixture flowing out from the space within the membrane area, and the geometry and resistance to flow of the space within the membrane area and the deformation of that space (examples for a membrane tube being ingoing pressure, outgoing pressure, internal diameter, and number and geometry of the curved sections of the membrane tube, and also the deformation of the curved sections) (H. N. Qi, C. T. Goudar, J. D. Michaels, H.-J. Henzler, G. N. Jovanovic, K. B. Konstantinov: “Experimental and Theoretical Analysis of Tubular Membrane Aeration for Mammalian Cell Bioreactors” Biotechnology Progress 19 (2003) pp. 1183-1189). In the case of membrane tubes, reduction of tube tension leads to increased deflection of the tubes during movement. Greater deflection of the tubes improves the flow around these and therefore improves the mass-transport coefficient. The tension is to be selected as a function of the nature of the application in such a way that the fixing of the membrane tubes is on the one hand stable over a long period of time, but that on the other hand the tubes preferably move within the flow and can undergo deflection amounting to, for example, a few millimetres.

Reduction of tube tension produces the problem of securing the membrane tubes on the winding arms. If tube tension is low, a large force acting on the membrane tubes could cause the membrane tubes to slip off the winding arms. To counter this problem, by way of example, the surface of the winding arms has an external screw thread. It is also possible, by way of example, to provide, externally on the winding arms, bars which inhibit external slippage of the tubes from the arms. Care has to be taken here that any unfinished edges of the screw thread do not damage the wound membrane tubes. The external thread on the winding arms of a star-shaped holder also provides the possibility of varying the winding of the tubes. By way of example, it is possible to use only every second or third screw-thread depression when winding the tubes. This permits establishment of defined separation between the individual membrane tubes.

The application WO2007098850(A1) gives further embodiments of membrane areas in the form of tubes fixed to the arms of a rotor and designed to execute a discontinuous movement.

The membrane area executing a discontinuous movement can be either completely or to some extent immersed in the culture medium. It is also possible to vary the immersion depth during the discontinuous movement.

The process according to the invention is versatile, e.g. in the culture of organisms, of human, animal or plant-derived cells, in the treatment of waste water, or in any other process in which deposits can form. It is preferably used in the culture of cell cultures which have a tendency towards agglomeration or adhesion to the bioreactor and to its elements, or where these are readily susceptible to agglomeration or adhesion of cells, cell debris or of substances. There are no disadvantageous effects here, for example on cell biology, e.g. in relation to apoptosis and cell cycle. Examples of cell cultures are BHK cells (Baby Hamster Kidney) for obtaining coagulation factors or CHO cells (Chinese Hamster Ovary) for obtaining therapeutic antibodies.

Use of a discontinuous, in particular a rotary oscillating, movement of the membrane area within the culture medium combines three functionalities:
1. The membrane area provides the necessary gas transfer and therefore the necessary supply of, for example, oxygen to the organisms, and also the necessary removal of gaseous products of metabolism of the organisms (in particular carbon dioxide).
2. The oscillating movement significantly improves mass transfer in comparison with a statically arranged membrane area onto which flow is directed by way of an additional agitator unit. There is no need for any additional agitator unit.
3. Surprisingly, the oscillating movement reduces formation of deposits and agglomerates, and this applies not only to deposits and agglomerates which become fixed to the membrane area but also to deposits and agglomerates which become fixed on other elements/areas within the bioreactor.

In addition to the discontinuous movement of the membrane area it is also possible to implement a discontinuous movement of one or more probes (pH probe, thermometer, oxygen-content-determining electrode and similar probes) in the bioreactor. Preference is given here to one or more probes being connected to the membrane area, if appropriate by way of a shared holder, so that membrane area and probe(s) are subjected to a shared/coupled movement. This is a more effective method of avoiding deposits on the probes.

### EXAMPLES

The invention is explained in more detail below by using examples, but is not restricted thereto.

### Example 1

### Apparatus for Conduct of the Process According to the Invention

FIG. 1 is a diagram of an example of an apparatus for conduct of the process according to the invention. The membrane area is formed via membrane tubes (1) which have been arranged vertically on a rotor shaft (2), perpendicularly with respect to the direction of rotation (3). Oxygen-containing gas can be pumped through the membrane tubes to supply organisms. The apparatus is preferably operated within a bioreactor (4). Complete immersion of the membrane area into the culture medium is preferred, in such a way that, during operation, the liquid surface (5) is above the membrane area. The apparatus can execute a rotary movement around the rotor shaft (2). It is preferable that it executes a rotary oscillating movement. The said movement leads firstly to improved supply to the organisms within the bioreactor and secondly to a markedly reduced tendency towards formation of deposits and agglomerates (in comparison with a static membrane area onto which flow is directed by an agitator unit).

FIG. 2 is a photograph of an apparatus which can accept membrane tubes. The upper part of the apparatus encompasses two concentric distributor rings for the input and discharge of gas. The exterior ring is mostly used for gas input, in order that the oxygen-rich gas passes first into the tube sections which are most distant from the rotor shaft, these being those to which the flow is most efficiently directed. In this example, each of the distributor rings has 16 nozzles, which permit supply to the membrane tube segments on the up to 16 rotor arms. This photograph shows the rotor with only 8 rotor arms mounted, and each of the 8 remaining possible rotor arms here would be mounted between those currently present. In this example, a membrane tube segment of length 57 m has been wound onto each rotor arm. If the rotor now rotates, the membrane tubes are moved through the fluid within the reactor and material thus flows onto them tangentially.

The apparatus shown in FIG. 2 for conduct of the process according to the invention serves for supply of gas to a cell-culture bioreactor with liquid capacity of about 200 L, where the internal diameter of the reactor is 510 mm and the height to diameter ratio is 2:1. The diameter of the central rotor shaft is 20 mm, and the external diameter of the rotor is 409 mm. The radius of the rotor arms in the depressed region within which the membrane tube has been conducted is 7.7 mm. Parallel depressed areas have been produced, separated by 3.65 mm, in order to inhibit slippage of the silicone membrane tubes, the internal diameter of which is 1.98 mm and the external diameter of which is 3.18 mm. The reason for the difference between 3.65 mm and 3.18 mm is the wish to retain space for volume increase (“blow-up”) of the membrane tubes even when they are subject to pressure (gauge pressure up to 1.5 bar), thus minimizing the pressure loss where the tube changes direction.

The rotor drive used can by way of example comprise a stepping motor with a maximum rotation rate of 2500 rpm, a stationary torque of 5.8 Nm and a gearbox reduction ratio of 1:12.

Table 1 lists examples for three sizes with the stated configuration, showing angular accelerations and maximum angular velocities, and also maximum velocities of the rotor arm ends, i.e. the fastest-moving points of the rotor.

| | | 15 L cell culture | 20 L cell culture | 200 L cell culture |
|---|---|---|---|---|
| | | system | system | system |
| Capacity | [L] | 12 | 20 | 200 |
| Angular acceleration | [rad s⁻²] | 4.3 | 3.7 | 0.75 |
| Angular retardation | [rad s⁻²] | 4.3 | 3.7 | 0.75 |
| Time for one movement | [ms] | 1250 | 2000 | 4000 |
| from starting to end-point | | | | |
| Angular displacement | [°] | 90 | 180 | 180 |
| Max. angular velocity | [rad s⁻¹] | 2.7 | 3.7 | 1.5 |
| Rotor diameter | [m] | 0.2 | 0.2 | 0.41 |
| Max. velocity of rotor ends | [m s⁻¹] | 0.27 | 0.37 | 0.31 |
| Tube length | [m] | 65 | 105 | 755 |
| Membrane area to capacity | [m² m⁻³] | 54.1 | 52.5 | 37.7 |
| ratio | | | | |
| Approx. power | [W m⁻³] | 12 | 11.1 | ~10* |
| consumption to capacity | | | | |
| ratio | | | | |

*measured in a geometrically similar model system

### Example 2

### Use of the Process According to the Invention for the Culture of HKB-11, a Human Hybrid Cell Line that has a Tendency Toward Adhesion

The process according to the invention was used by way of example during the culture of human cell line HKB-11 for producing blood coagulation factor VIII (Mei, Baisong et al., “Expression of Human Coagulation Factor VIII in a Human Hybrid Cell Line”, HKB11, Molecular Biotechnology. 34(2):165-178, October 2006). This cell line has very high tendency to form aggregates.

Alongside this, the same cell line was cultured in a reference process (not according to the invention), in order to permit comparison of the processes.

The process according to the invention was conducted in a 15 L bioreactor from Applikon. The bioreactor had a rotor, on which there was a membrane area in the form of silicone tubes (SILASTIC RX 50 Medical Grade Tubing Special, 0.078 in. (1.98 mm) ID×0.125 in. (3.18 mm) OD (500 ft roll, Dow Corning)). The membrane tubes had been secured to the 8 arms of the rotor, which had been attached in the manner of a star around a rotor shaft. The total length of membrane tubes was 58.7 m (48.8 m² of membrane surface per m³ of reactor volume for 12 L capacity), and there was no winding on the two innermost rows of the rotor arms. A full winding would have given a total length of 65 m of membrane tube (54.1 m² of membrane surface per m³ of reactor volume for 12 L capacity). A servomotor (23S21, Jenaer Antriebstechnik, Jena, Germany) with stationary torque of 0.9 Nm with a flanged joint to a planetary drive with a gearbox reduction ratio of 1:12 was available to provide discontinuous motion of the rotor. The following reference gives information on the human HKB cell line used: Mei, Baisong et al., “Expression of Human Coagulation Factor VIII in a Human Hybrid Cell Line”, HKB11, Molecular Biotechnology. 34(2):165-178, October 2006.

By way of the membrane area (membrane tubes), the cells were supplied with oxygen and freed from carbon dioxide. Gas throughput was 1 standard litre per hour. The gas flowing through the membrane tubes on the 8 rotor arms is collected again at the end of the membrane tubes and conducted through a flexible tube to the top cover of the bioreactor, where the counterpressure at the gas outlet varies from 5 to 15 psig. This permits control of gas transfer properties. An air flow of 1 standard litre per hour per aeration nozzle and ventilation nozzle was passed continuously through the head space of the fermenter during the culture process. Information on the structure of the system for continuous cell culture is available in WO2003/020919 A1.

According to the invention, the membrane area within the culture medium was subjected to a rotary oscillating movement. The sequence of movement was as follows: starting from one of the points of reversal of rotational direction, the membrane area was accelerated for a period of 400 ms with a constant angular acceleration of 11 rad s⁻², and was then retarded for the same period with a numerically identical angular acceleration, thus again becoming stationary after 800 ms. The displacement angle is 90°. Power consumption amounts to about 56 W m⁻³. The maximum velocity of the rotor ends is about 0.44 m s⁻¹.

The process according to the invention is hereafter termed DMA process (Dynamic Membrane Aeration), and the corresponding apparatus for conduct of the process according to the invention is termed DMA reactor.

The reference process was likewise conducted in a 15 L bioreactor (reference reactor) of identical structure from Applikon. This had a static membrane area and an anchor stirrer. The static membrane area encompassed a length of 49.6 m of the abovementioned silicone tube (corresponding to 41.3 m² of membrane surface area per m³ of reactor capacity) (the same make of silicone tube was used for the DMA system and for the reference system). The flow rate through the membrane tubes was 0.5 standard litre per hour. The anchor stirrer, designed in-house, served to direct flow onto the membrane area in order to improve transfer of materials (supply of oxygen, dissipation of carbon dioxide). The anchor stirrer was operated at a constant rotation rate of 150 rpm (corresponding to about 165 W m⁻³). This high stirrer rotation rate, or this high power consumption, is usually avoided for reasons of cell damage and undesired formation of by-products, but was necessary in order to avoid/restrict cell agglomeration and formation of deposits.

Information on the structure of the system for continuous cell culture, and on the cell separator, are again found in WO2003/020919A1.

An adequate amount of cell inoculum that was used for the inoculation of the reference system was grown in advance in shaker flasks. The 15 L DMA reactor was inoculated with cells from the reference system, giving comparability of the two systems in respect of shared source of cells and also in respect of identical cell age, with the exception of the slight time difference. Examples of the inoculation cell densities can be found in FIG. 3 and FIG. 4.

Specimens were taken daily from the bioreactor and from the harvest stream, both from the DMA process and from the reference process, and these were analysed for cell density, vitality, aggregation rates, off-line pH, concentration of dissolved oxygen and of dissolved carbon dioxide, concentration of glucose, lactate, glutamine, glutamate, ammonium, and LDH and titer (blood coagulation factor VIII (rFVIII)).

FIG. 3 shows the growth of density of living cells as a function of time in a first cell cultivation process (a) in the DMA process and (b) in the reference process. In each case, the density cd of living cells has been plotted in the unit [10⁶ cells mL⁻¹] against time t in the unit [days]. Cell density was determined by using a CEDEX system (Innovatis GmbH, Bielefeld, Germany). In order to minimize the effect of cell agglomeration and determine a cell density that was as representative as possible, a prior pipetting process was used, thus substantially breaking up the cell agglomerates by virtue of the shear forces in the pipette. FIG. 3(b) shows a reduction in cell density to about 10×10⁶ cells mL⁻¹ after 53 days. Deposits on the membrane tubes caused this, and appear to have reduced oxygen ingress. These deposits were not observed in the DMA process; here, it was possible to maintain high cell density over the entire period of observation.

After the first cell-cultivation process, the bioreactor of the DMA process was washed solely with medium (the formulation of which is confidential). Deposits remained on the sensors and on the membrane area here. A second cell-cultivation process was then conducted with a fresh input of cells. The procedure served to simulate a long-term cultivation process.

FIG. 4 shows the growth of density of living cells as a function of time in the second cell cultivation process (a) in the DMA process and (b) in the reference process. In each case, the density cd of living cells has been plotted in the unit [10⁶ cells mL⁻¹] against time t in the unit [days].

Cell density of more than 15×10⁶ cells mL⁻¹ was achieved in just under 7 days in the DMA process, and was maintained. The reference process did not achieve that cell density; the production rate was correspondingly lower.

In both cell culture processes, the DMA process therefore exhibited higher cell density and therefore a higher production rate than the reference process. The reason has been demonstrated to be the reduced tendency towards formulation of deposits in the DMA process in comparison with the reference process.

In summary, the example revealed the following advantages of the process according to the invention in comparison with the reference process described:
Increased oxygen ingress; during the entire culture time, cell density was on average higher in the DMA process than in the reference process.
The amount of deposits observed in the DMA process was smaller, not only on the membrane tubes but also on the non-moving parts of the bioreactor and on the probes.
The DMA process used about one third of the power consumption of the reference system to achieve comparable flow conditions (based on shear rate).
The DMA process displayed no adverse effects on cell biology (apoptosis and cell cycle).

### BRIEF DESCRIPTION OF THE DRAWINGS

FIG. 1: Diagram of a rotary oscillating movement for supplying gas to, and removing gas from, liquids by means of a membrane area in a container.

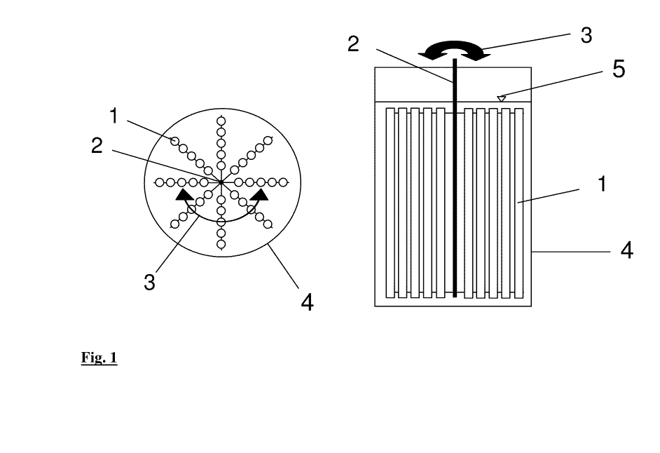

FIG. 2: Photograph of an apparatus to which a membrane area can be applied: membrane tubes have been wound to the rotor arms arranged in the form of a star.

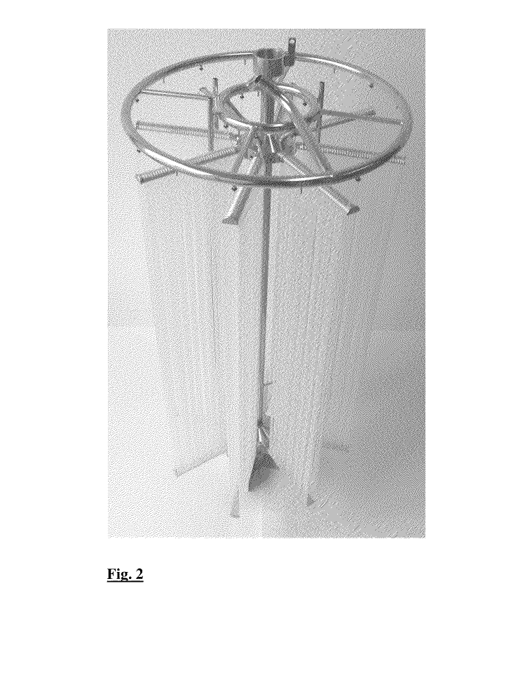

FIG. 3: Graph of density of living cells as a function of time in a first cell cultivation process (a) in the DMA process and (b) in the reference process. In each case, the density cd of living cells has been plotted in the unit [10⁶ cells mL⁻¹] against time t in the unit [days].

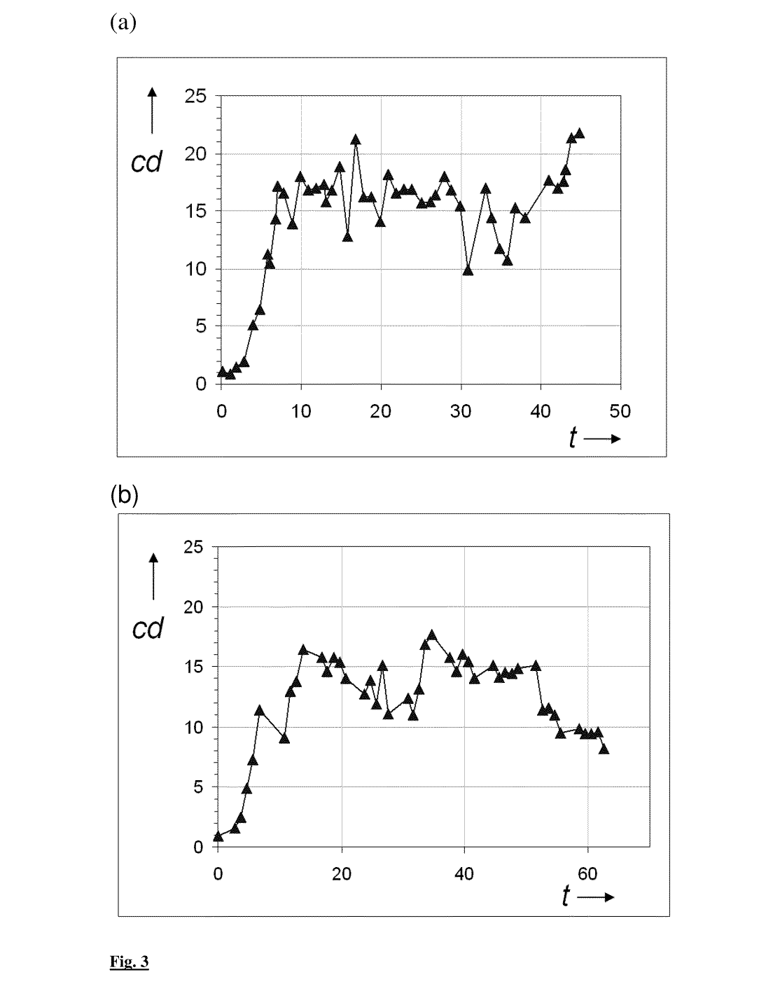

FIG. 4: Graph of density of living cells as a function of time in a second cell cultivation process (a) in the DMA process and (b) in the reference process. In each case, the density cd of living cells has been plotted in the unit [10⁶ cells mL⁻¹] against time t in the unit [days].

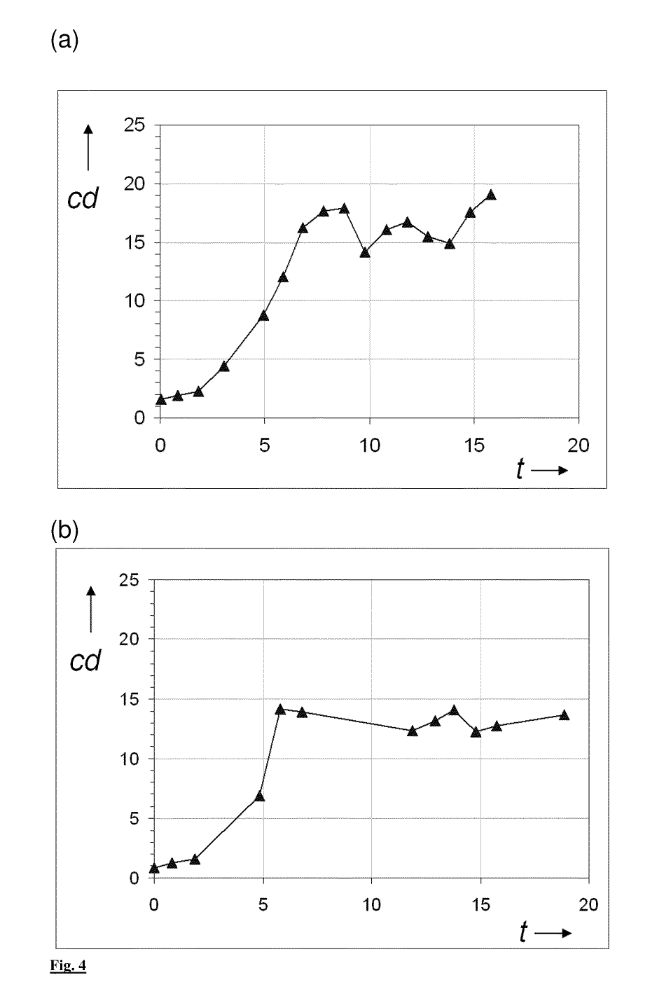

### Key:

1 Membrane tube
2 Rotor shaft
3 Direction of rotation
4 Bioreactor
5 Liquid level

1. Process for reducing the amount of deposits on probes during the culture of cells and organisms in a bioreactor, where a membrane area immersed in the culture medium for purposes of gas transfer executes a discontinuous movement, wherein, in addition to the discontinuous movement of the membrane area, there is also a discontinuous movement of one or more probes.

2. Process according to claim 1, wherein the membrane area executes a movement with reversal of movement.

3. Process according to claim 1, wherein the membrane area executes a rotary oscillating movement.

4. Process according to any of claims 1, wherein the movement encompasses a periodic sequence of acceleration and retardation between two points of reversal of movement.

5. Process according to any of claims 1, wherein the membrane area has been formed from one or more membrane tubes.

6. Process according to claim 5, wherein the membrane area in the form of one or more membrane tubes has been attached to rotor arms attached in the form of a star to a rotor shaft, and is impacted by tangential flow as a consequence of the discontinuous movement.

7. Process according to claim 1, wherein there is/are one or more probes connected to the membrane area.

8. Process according to claim 7, wherein there is/are one or more probes connected to the membrane area by way of a shared holder.

9. Process according to claim 1 for the culture of cells or organisms.

10. Bioreactor comprising a discontinuously moveable membrane area immersed in the culture medium, wherein there is/are one or more probes connected to the membrane area.

11. Bioreactor according to claim 10, where the connection between the one or more probes and the membrane area is provided by way of a shared holder.
